(19) United States
(12) Patent Application Publication
(10) Pub. No.: US 2016/0071061 A1
(43) Pub. Date: Mar. 10, 2016

(54) SYSTEM AND METHOD FOR AUTOMATED CREATION AND VALIDATION OF PHYSICAL ABILITY TESTS FOR EMPLOYMENT ASSESSMENT

(71) Applicants: BIDDLE CONSULTING GROUP, INC., Folsom, CA (US); WORKSTEPS, INC., Austin, TX (US)

(72) Inventors: Dan Biddle, Folsom, CA (US); Larry Feeler, Austin, TX (US)

(21) Appl. No.: 14/481,738

(22) Filed: Sep. 9, 2014

(51) Int. Cl. G06Q 10/10; A61B 5/00

(52) U.S. Cl. CPC G06Q 10/1053; A61B 5/7275

(57) ABSTRACT

A system and method for the automated creation and validation of physical ability tests for employment assessment is disclosed. A particular embodiment includes: enabling, by use of a computing device, a user to edit and configure a plurality of physical ability test (PAT) event parameters including: environment parameters, restrictions, requirements, and risk parameters; enabling, by use of the computing device, the user to identify and describe essential physical tasks of a job description being tested; automatically creating a proposed PAT event based on the PAT event parameters and the essential physical tasks; automatically generating a validation survey corresponding to the proposed PAT event; sending the validation survey with an invitation to a set of job experts for feedback in a data network communication; and creating a test parameter report and a validation report based on the PAT event parameters, the essential physical tasks, and the feedback from the set of job experts.

### COPYRIGHT

A portion of the disclosure of this patent document contains material that is subject to copyright protection. The copyright owner has no objection to the facsimile reproduction of the patent document or the patent disclosure, as it appears in the Patent and Trademark Office patent files or records, but otherwise reserves all copyright rights whatsoever. The following notice applies to the software and data as described below and in the drawings that form a part of this document: Copyright 2013-2014 Biddle Consulting Group, Inc., All Rights Reserved.

### TECHNICAL FIELD

This patent application relates to computer-implemented software systems, according to one embodiment, and more specifically to a system and method for the automated creation and validation of physical ability tests for employment assessment.

### BACKGROUND

For years, medical and vocational evaluators have been testing and otherwise attempting to evaluate dexterity and other physical qualifications of applicants and workers to assist in safe and productive placement in specific jobs. Most of these tests measure one or more of the physical demands and aptitudes of jobs for which some kind of physical ability is required. However, it has been particularly difficult to ensure that the tests of physical ability are properly aligned with the actual physical skills needed by a worker in a particular job. Moreover, federal and state law requires a level of validation and documentation that a particular physical ability test is appropriate for a particular job offering. Current technology has been unable to offer an automated way to develop consistent, equitable, and compliant physical ability tests for employment assessment.

### SUMMARY

In various example embodiments, a system and method for the automated creation and validation of physical ability tests for employment assessment is disclosed. In the various example embodiments described herein, a computer-implemented tool or application (app) is described to automate the physical ability test event creation using the parameters and values required for a particular job offering (e.g., required distances, heights, weights, forces, etc.). As described in more detail herein, the example embodiment can create an online physical ability test event definition, a related validation survey, and necessary documentation that addresses the Federal Uniform Guidelines on Employee Selection Procedures, among other employment assessment requirements. The example embodiments can generate a physical ability test event and automatically create legally-defensible validation documents.

The various example embodiments described herein can provide a variety of unique features not present in related conventional systems. These unique features include at least: 1) dynamic lift reserve calculation, and 2) dynamic weight variation. These features are described in more detail below.

The various example embodiments described herein can determine whether or not a physical ability test event is valid based upon the test parameters, the ratings, and an automatically determined threshold for validity. The example embodiments can also automatically generate a report to document the validity of the physical ability test event (Validation Report). This report contains all the required documentation as set forth by the Federal Uniform Guidelines on Employee Selection Procedures, section 15C. The example embodiments can also automatically generate a Test Parameter Report, which allows a job analyst to print and execute a “test-center ready” list of the physical ability test events along with any additional notes.

The various example embodiments described herein can assist a job analyst, an employer, or the like to design, generate, and execute a physical ability test (PAT) event for a particular job title/description that is appropriate, effective, and legally compliant. The various embodiments use an appropriate foundation to design the PAT event based on dynamic job analysis or existing job-related data. The various embodiments ensure that job duties tested are linked to KSA (Knowledge, Skills, and Abilities) requirements and applicable physical job requirements. The example embodiments can create a physical ability job simulation test event that addresses the Federal Uniform Guidelines on Employee Selection Procedures and other employment assessment requirements. The example embodiments can generate a standardized, content-valid, physical ability test that is effective and legally defensible. The example embodiments can also generate a related custom test validation document and a test parameter document. The automatically generated physical ability test (PAT) is built with standardized events for: pushing, pulling/dragging, gripping/pinching, lifting without carrying, lifting with carrying, climbing, ascending/descending steps, and walking, among other physical skills being tested.

As described in more detail below and in the accompanying drawings, the example embodiments enable a user to manipulate a menu of data objects, in a computer-generated user interface, to: import, add, or modify job duties; import, add, or modify work environment parameters; import, add, or modify physical restriction parameters; import, add, or modify vision requirements; import, add, or modify identified risks; import, add, or modify physical characteristics; import, add, or modify physical ability parameters; send validation survey invites to appropriate job experts; generate various reporting documents; and manage a variety of other operations related to the design, generation, and execution of a physical ability test (PAT) related to particular job description.

As configured and directed by the user via the computer-generated user interface, the example embodiments described herein can automatically create the physical ability test events and the validation survey for which job experts provide ratings. The various embodiments can manage the publication of the validation survey to a defined set of job experts and can manage the retrieval of validation survey feedback from the job experts. After the validation survey is completed and the job expert feedback is retrieved, an example embodiment can dynamically and automatically generate a validation report including the feedback from the defined set of job experts. An example embodiment can also generate a test parameter report, which allows the job analyst to execute the physical ability test event and provide any additional notes or comments regarding the physical ability test (PAT) event.

As described in more detail herein and in the accompanying drawings, the example embodiments realize a number of important benefits. The example embodiments can dynamically generate a physical ability test event and automatically create related legally-defensible test validation and parameter reports that are fully documented and compliant with applicable legal requirements. The example embodiments can dynamically produce a job simulation physical ability test event that is linked to the content and requirements of a particular job description. Moreover, the job content is linked to the job analysis, which links back to the source. In various embodiments described herein, the PAT development process is easy to use and driven by the needs of organizations of all sizes (SME-driven). In one embodiment, the system is web-based, tablet (e.g., iPad) compatible, and supported for cross-platform use. The documentation produced by the system, including the test validation and parameter reports, is designed to survive audits and legal challenges. Various example embodiments are described in more detail below and in the accompanying drawings.

### BRIEF DESCRIPTION OF THE DRAWINGS

The various embodiments are illustrated by way of example, and not by way of limitation, in the figures of the accompanying drawings in which:

FIG. 1 illustrates an example embodiment of a process used in a system for enabling users to automatically create and validate physical ability tests for employment assessment;

FIG. 2 illustrates an example embodiment of a system for enabling computer users to automatically create and validate physical ability tests for employment assessment;

FIG. 3 illustrates the components of the system databases used in an example embodiment;

FIG. 4 illustrates a detail of the job analysis module in an example embodiment;

FIG. 5 illustrates a detail of the physical ability test (PAT) validation module in an example embodiment;

FIGS. 6 through 17 illustrate example user interface screen snapshots of example embodiments;

FIGS. 18 through 21 illustrate a sample Test Validation Report automatically created by the PAT development system of an example embodiment;

FIGS. 22 through 23 illustrate a sample Test Parameter Report automatically created by the PAT development system of an example embodiment;

FIG. 24 illustrates another example embodiment of a networked system in which various embodiments may operate;

FIG. 25 illustrates a processing flow diagram that illustrates an example embodiment of a PAT development system as described herein; and

FIG. 26 shows a diagrammatic representation of a machine in the example form of a computer system within which a set of instructions when executed may cause the machine to perform any one or more of the methodologies discussed herein.

### DETAILED DESCRIPTION

In the following description, for purposes of explanation, numerous specific details are set forth in order to provide a thorough understanding of the various embodiments. It will be evident, however, to one of ordinary skill in the art that the various embodiments may be practiced without these specific details.

In the various embodiments described herein, a system and method for the automated creation and validation of physical ability tests for employment assessment is disclosed. In various embodiments, a software application program is used to enable the development and validation of physical ability tests for employment assessment on the display screen of a computer system, including mobile devices. As described in more detail below, the computer or computing system on which the described embodiments can be implemented can include personal computers (PCs), portable computing devices, laptops, tablet computers, personal digital assistants (PDAs), personal communication devices (e.g., cellular telephones, smartphones, or other wireless devices), network computers, set-top boxes, consumer electronic devices, or any other type of computing, data processing, communication, networking, or electronic system.

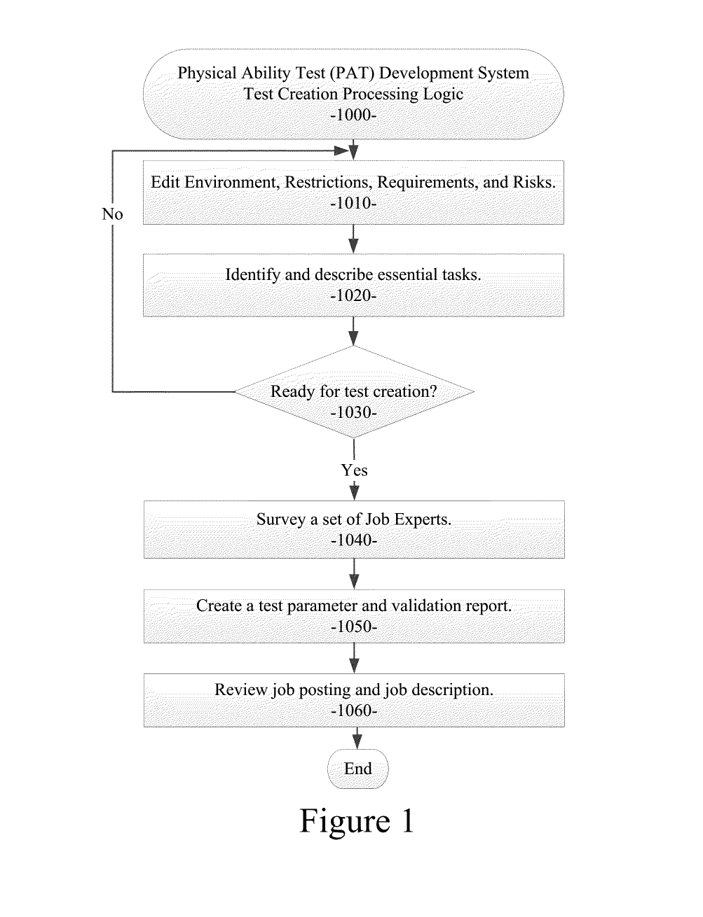

Referring now to FIG. 1, in an example embodiment, a process used in a system for enabling users to automatically create and validate physical ability tests for employment assessment is disclosed. In a particular example embodiment, processing logic is provided as part of a Physical Ability Test (PAT) Development System. In the example embodiment, the processing logic can enable the user to edit and configure various PAT event test parameters including, environment parameters, restrictions, requirements, and risk parameters (processing block 1010). The processing logic can enable the user to identify and describe essential physical tasks of the job description being tested (processing block 1020). Once the PAT event test parameters are configured and the essential physical tasks are identified and described, the PAT Development System of an example embodiment can create a proposed PAT event (processing block 1030). After the proposed PAT event is created, the PAT Development System of an example embodiment can automatically generate a validation survey corresponding to the proposed PAT event to survey a set of job experts for feedback related to the proposed PAT event and the applicability of the test event to the job description for which the proposed PAT event is being created (processing block 1040). The job expert feedback is an important component supporting the legal compliance of the physical ability test event with applicable regulations and guidelines. The PAT Development System can also create a test parameter report and a validation report to document the compliance of the physical ability test event (processing block 1050). Finally, the PAT Development System can review and/or generate a related job posting and job description to validate the consistent and compliant presentation of the job listing and related physical ability test to potential job applicants (processing block 1060).

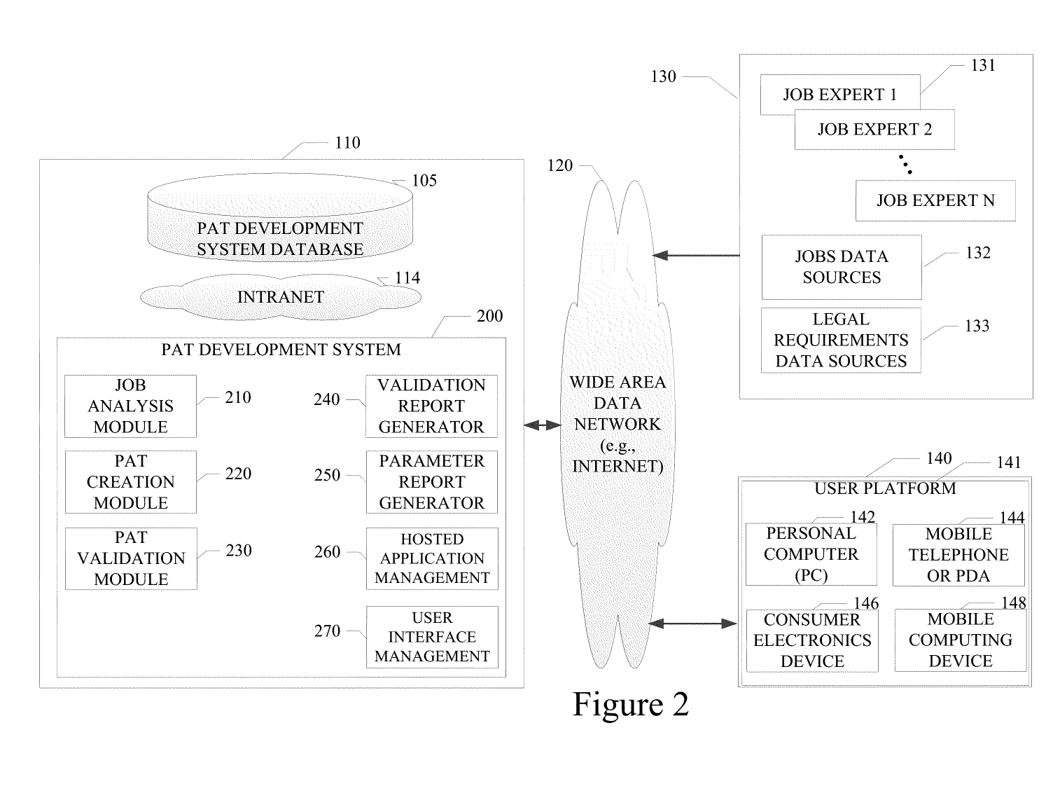

Referring now to FIG. 2, in an example embodiment, a system for enabling users to automatically create and validate physical ability tests for employment assessment is disclosed. In various example embodiments, an application or service, typically provided by or operating on a host site (e.g., a website) 110, is provided to simplify and facilitate the downloading or hosted use of the physical ability test (PAT) development system 200 of an example embodiment. In a particular embodiment, the PAT development system 200 can be downloaded from the host site 110 by a user at a user platform 140. Alternatively, the PAT development system 200 can be hosted by the host site 110 for a networked user at a user platform 140. The details of the PAT development system 200 for an example embodiment are provided below.

Referring again to FIG. 2, the PAT development system 200 can be in data network communication with a plurality of external data sources 130. The external data sources 130 can include networked job expert computing systems 131, jobs data sources 132, legal requirement data sources 133, and a variety of other external networked data sources 130. In a particular embodiment, jobs-related data, job expert data including validation survey data, and legal requirement data associated with job descriptions can be retrieved from the various external data sources 130 and used by the PAT development system 200 on the host site 110 to automatically construct PAT events and related documents based on all the information the PAT development system 200 has retrieved from the various external data sources 130 and stored in its database 105. The data retained by database 105 in an example embodiment is detailed below.

One or more of the external data sources 130 can be provided by one or more third party providers operating at various locations in a network ecosystem. It will be apparent to those of ordinary skill in the art that external data sources 130 can be any of a variety of networked third party providers as described in more detail herein. In a particular embodiment, a source list maintained at the host site 110 can be used as a summary or list of all external data sources 130, which users or the host site 110 may visit/access and from which users or the host site 110 can obtain jobs-related data. The host site 110, external data sources 130, and user platforms 140 may communicate and transfer data and information in the data network ecosystem shown in FIG. 2 via a wide area data network (e.g., the Internet) 120. Various components of the host site 110 can also communicate internally via a conventional intranet or local area network (LAN) 114.

Networks 120 and 114 are configured to couple one computing device with another computing device. Networks 120 and 114 may be enabled to employ any form of computer readable media for communicating information from one electronic device to another. Network 120 can include the Internet in addition to LAN 114, wide area networks (WANs), direct connections, such as through a universal serial bus (USB) port, other forms of computer-readable media, or any combination thereof. On an interconnected set of LANs, including those based on differing architectures and protocols, a router and/or gateway device acts as a link between LANs, enabling messages to be sent between computing devices. Also, communication links within LANs typically include twisted wire pair or coaxial cable, while communication links between networks may utilize analog telephone lines, full or fractional dedicated digital lines including T1, T2, T3, and T4, Integrated Services Digital Networks (ISDNs), Digital Subscriber Lines (DSLs), wireless links including satellite links, or other communication links known to those of ordinary skill in the art. Furthermore, remote computers and other related electronic devices can be remotely connected to either LANs or WANs via a wireless link, WiFi, Bluetooth, satellite, or modem and temporary telephone link.

Networks 120 and 114 may further include any of a variety of wireless sub-networks that may further overlay stand-alone ad-hoc networks, and the like, to provide an infrastructure-oriented connection. Such sub-networks may include mesh networks, Wireless LAN (WLAN) networks, cellular networks, and the like. Networks 120 and 114 may also include an autonomous system of terminals, gateways, routers, and the like connected by wireless radio links or wireless transceivers. These connectors may be configured to move freely and randomly and organize themselves arbitrarily, such that the topology of networks 120 and 114 may change rapidly and arbitrarily.

Networks 120 and 114 may further employ a plurality of access technologies including 2nd (2G), 2.5, 3rd (3G), 4th (4G) generation radio access for cellular systems, WLAN, Wireless Router (WR) mesh, and the like. Access technologies such as 2G, 3G, 4G, and future access networks may enable wide area coverage for mobile devices, such as one or more of client devices 141, with various degrees of mobility. For example, networks 120 and 114 may enable a radio connection through a radio network access such as Global System for Mobile communication (GSM), General Packet Radio Services (GPRS), Enhanced Data GSM Environment (EDGE), Wideband Code Division Multiple Access (WCDMA), CDMA2000, and the like. Networks 120 and 114 may also be constructed for use with various other wired and wireless communication protocols, including TCP/IP, UDP, SIP, SMS, RTP, WAP, CDMA, TDMA, EDGE, UMTS, GPRS, GSM, UWB, WiFi, WiMax, IEEE 802.1 lx, and the like. In essence, networks 120 and 114 may include virtually any wired and/or wireless communication mechanisms by which information may travel between one computing device and another computing device, network, and the like. In one embodiment, network 114 may represent a LAN that is configured behind a firewall (not shown), within a business data center, for example.

The external data sources 130 may include any of a variety of providers of network transportable digital data. The network transportable digital data can be transported in any of a family of file formats and associated mechanisms usable to enable a host site 110 and a user platform 140 to receive data from a jobs-related data source 130 over a network 120. In one embodiment, the file format can be a MICROSOFT® Excel spreadsheet format or a CSV (Comma Separated Values) format; however, the various embodiments are not so limited, and other file formats and transport protocols may be used. For example, data formats other than Excel or CSV or formats other than open/standard formats can be supported by various embodiments. Any electronic file format, such as Microsoft® Access Database Format (MDB), Portable Document Format (PDF), audio (e.g., Motion Picture Experts Group Audio Layer 3—MP3, and the like), video (e.g., MP4, and the like), and any proprietary interchange format defined by specific sites can be supported by the various embodiments described herein. Moreover, a jobs-related data source 130 may provide more than one jobs-related data set and/or more than one validation survey from a job expert.

In a particular embodiment, a user platform 140 with one or more client devices 141 enables a user to access PAT data and documents from the PAT development system 200 via the host 110 and network 120. Client devices 141 may include virtually any computing device that is configured to send and receive information over a network, such as network 120. Such client devices 141 may include portable devices 144, such as, cellular telephones, smart phones, display pagers, radio frequency (RF) devices, infrared (IR) devices, global positioning devices (GPS), Personal Digital Assistants (PDAs), handheld computers, wearable computers, tablet computers, integrated devices combining one or more of the preceding devices, and the like. Client devices 141 may also include other computing devices, such as personal computers 142, multiprocessor systems, microprocessor-based or programmable consumer electronics, network PC's, and the like. Client devices 141 may also include other processing devices, such as consumer electronic (CE) devices 146 and/or mobile computing devices 148, which are known to those of ordinary skill in the art. As such, client devices 141 may range widely in terms of capabilities and features. For example, a client device configured as a cell phone may have a numeric keypad and a few lines of monochrome LCD display on which only text may be displayed. In another example, a web-enabled client device may have a touch sensitive screen, a stylus, and several lines of color LCD display in which both text and graphics may be displayed. Moreover, the web-enabled client device may include a browser application enabled to receive and to send wireless application protocol messages (WAP), and/or wired application messages, and the like. In one embodiment, the browser application is enabled to employ HyperText Markup Language (HTML), Dynamic HTML, Handheld Device Markup Language (HDML), Wireless Markup Language (WML), WMLScript, JavaScript, EXtensible HTML (xHTML), Compact HTML (CHTML), and the like, to display and/or send digital information. In other embodiments, mobile devices can be configured with applications (apps) with which the functionality described herein can be implemented.

Client devices 141 may also include at least one client application that is configured to receive and process jobs-related data or/or PAT event data from another computing device via a wired or wireless network transmission. The client application may include a capability to provide and receive textual data, graphical data, video data, audio data, and the like. Moreover, client devices 141 may be further configured to communicate and/or receive a message, such as through a Short Message Service (SMS), direct messaging (e.g., Twitter), email, Multimedia Message Service (MMS), instant messaging (IM), internet relay chat (IRC), mIRC, Jabber, Enhanced Messaging Service (EMS), text messaging, Smart Messaging, Over the Air (OTA) messaging, or the like, between another computing device, and the like.

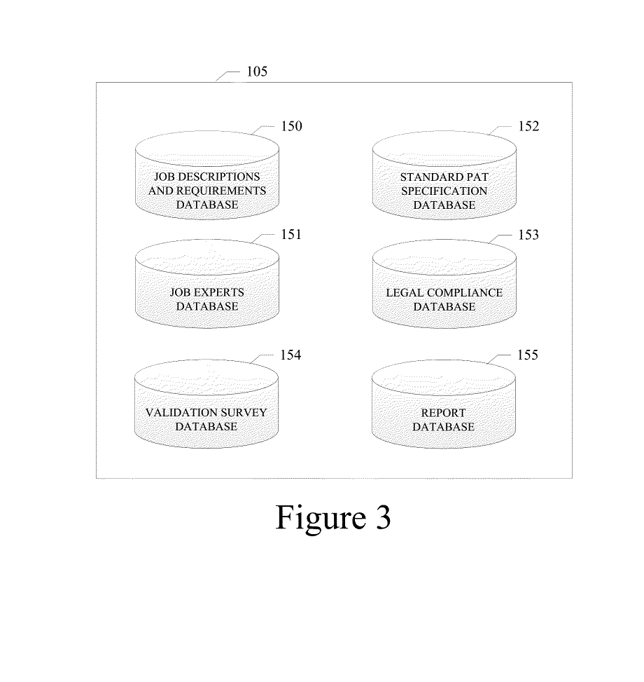

Referring again to FIG. 2, the PAT development system 200 for an example embodiment is shown to include a PAT development system database 105. Referring now to FIG. 3, the components of database 105 are illustrated for an example embodiment. Each of these database 105 components is described in detail below.

Job Descriptions and Requirements Database 150—In an example embodiment, job description and job requirements data for a variety of specific types of jobs or job categories can be obtained and stored in this database. The job description and job requirements data can include information specifying the environment, restrictions, requirements, and risks associated with particular job types or job categories. In particular, the job description and job requirements data can include information specifying the types of locations or environments in which the job is typically performed, the capabilities, physical abilities, or restrictions (e.g., vision) a qualified job applicant must have, the types of physical activities or tasks required for a particular job type or job category, the amount of weight to be moved, the distances traveled, the types of equipment used, the amount of force or power needed, the quantity of repetition, the time periods over which the physical activities are performed, the types of risks associated with the job type or job category, and a variety of other information, data, parameters, and/or metrics that define the types of physical activity or capabilities needed for particular jobs or job categories. This data can be pre-loaded from existing datasets or downloaded from various job data sources 132. The job description and job requirements data can be retained in database 150 and used by the PAT development system 200 to assist a user in automatically creating and executing a physical ability test for a particular job offering.

Job Experts Database 151—In an example embodiment, a physical ability test automatically created for a particular job offering by the PAT development system 200 can be vetted and validated by a set of qualified job experts with an expertise in the job category associated with the particular job offering. Information related to these job experts can be retained in the database 151. For example, the retained job expert data can include the names, experience profiles, qualified job categories, contact information, and other information associated with the pool of qualified job experts who can validate a proposed physical ability test for a particular job offering. Once a proposed physical ability test is created for a particular job offering by the PAT development system 200, the proposed physical ability test can be automatically published for validation to the set of qualified job experts 131 via network 120.

Standard PAT Specification Database 152 —In an example embodiment, the PAT development system 200 can be used to enable a user to create physical ability tests for particular job offerings as described herein. Over time, a set of validated physical ability tests for particular job types or categories can be created and stored in database 152. As a result, the user may avoid creating a new physical ability test for particular job offering from scratch if a previously-created compliant physical ability test is already present in the database 152. Additionally, compliant physical ability tests for particular job types or categories can be retrieved from jobs data sources 132 and stored in database 152.

Legal Compliance Database 153—In most cases, a physical ability test created for a particular job offering must comply with a variety of legal requirements imposed by various regulatory agencies, such as the U.S. Dept. of Labor, the Occupational Safety and Health Administration (OSHA), various state and local governmental, judicial, and administrative agencies. Additionally, physical ability tests created for particular job offerings should comply with a variety of standards or guidelines published by various non-governmental organizations, standards bodies, non-profits, corporate entities, and other organizations interested in promoting fairness, equality, and transparency in employment practices. Information and data associated with these various legal and ethical employment compliance standards can be obtained from various legal requirement data sources 133 via network 120 and stored in database 153. The employment compliance standards data can be retained in database 153 and used by the PAT development system 200 to assist a user in automatically creating and executing a legally compliant physical ability test for a particular job offering.

Validation Survey Database 154—Once a proposed physical ability test is created for a particular job offering by the PAT development system 200, the proposed physical ability test can be automatically published for validation to the set of qualified job experts 131 via network 120. As part of the validation, the set of qualified job experts 131 can be offered a validation survey to record their assessment of the proposed physical ability test. The set of qualified job experts can return their completed validation surveys via network 120. These validation surveys and meta data defining their identity and provenance can be stored in database 154. These validation surveys can be appropriately configured and formatted to comply with the associated legal requirements defined in the Legal Compliance Database 153.

Report Database 155—Once a proposed physical ability test is created for a particular job offering by the PAT development system 200, the proposed physical ability test can be automatically published for validation as a validation report to the set of qualified job experts 131. The validation report can be retained in database 155. Additionally, once the proposed physical ability test is validated by the set of qualified job experts 131, a test parameter report can be automatically generated by the PAT development system 200 to assist the job analyst in administering or executing the validated physical ability test. The test parameter report can also be retained in database 155.

Referring again to FIG. 2, host site 110 of an example embodiment is shown to include a physical ability test (PAT) development system 200. PAT development system 200 includes: job analysis module 210, physical ability test (PAT) creation module 220, physical ability test (PAT) validation module 230, validation report generator 240, parameter report generator 250, hosted application management 260, and user interface management 270. Each of these modules can be implemented as software components executing within an executable environment of PAT development system 200 operating on host site 110 or user platform 140. Each of these modules of an example embodiment is described in more detail below in connection with the figures provided herein.

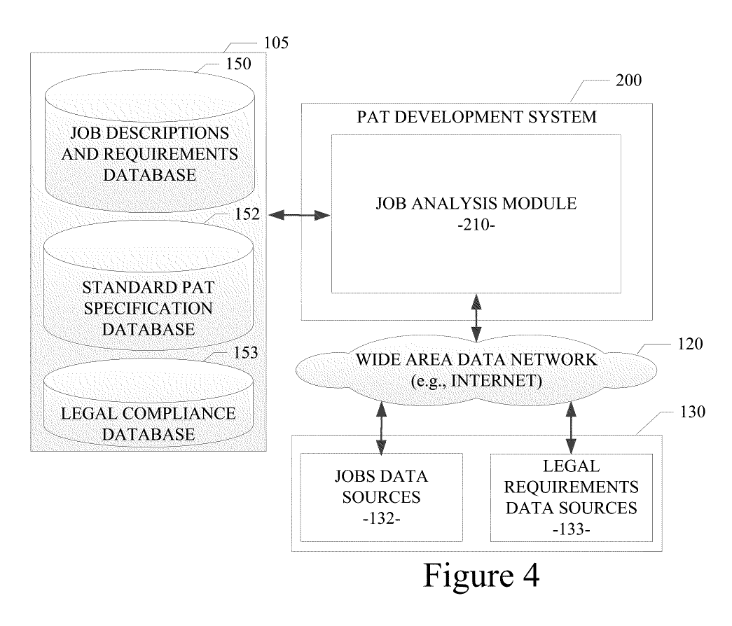

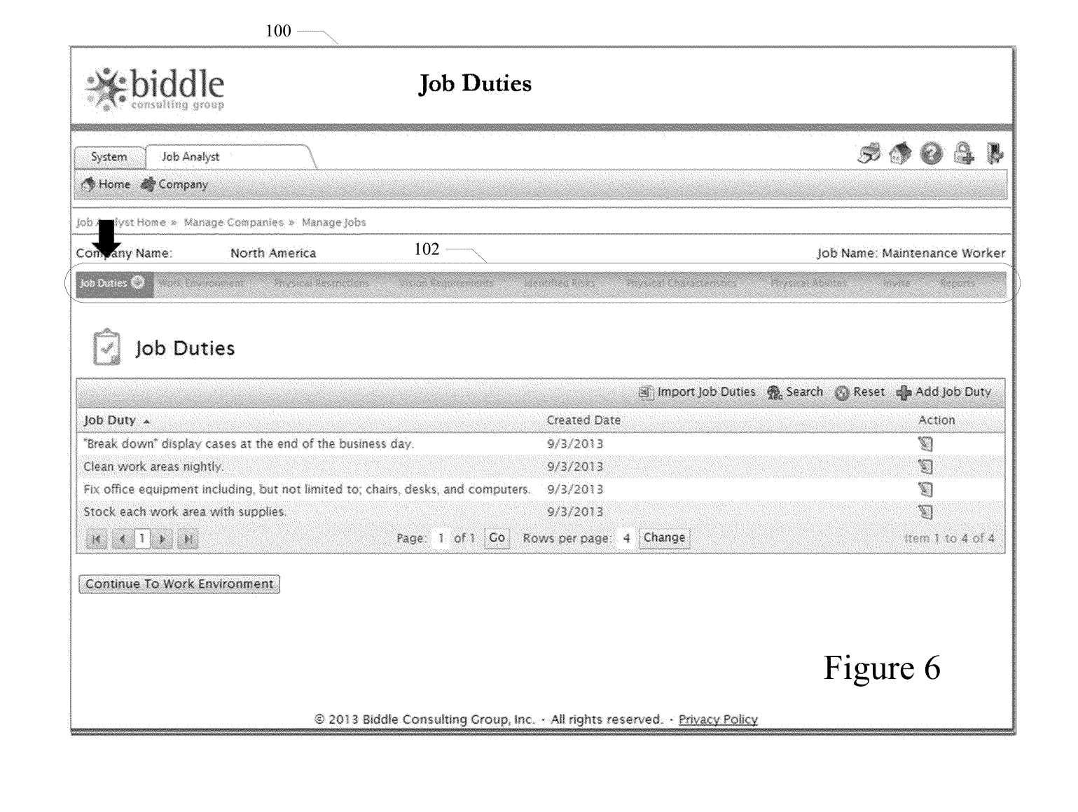

Referring now to FIG. 4, a detail of the job analysis module 210 in an example embodiment is illustrated. As shown, job analysis module 210 is in data communication via network 120 with a plurality of external data sources 130, one or more portions of database 105, and the other processing modules of the PAT development system 200 (not shown in FIG. 4). In general, the job analysis module 210 is responsible for collecting and processing job-related data (e.g., job descriptive data, legal compliance data, etc.) from a variety of external data sources 130. In an example embodiment, the processing logic of the job analysis module 210 can enable the user to retrieve, edit, and configure various job-related and/or PAT event test data and parameters including, environment parameters, restrictions, requirements, and risk parameters associated with a particular job description for which a physical ability test event is being created. The processing logic of the job analysis module 210 can further enable the user to identify and describe essential physical tasks of the job description being tested. The job analysis module 210 can further enable the user to specify job duties, describe the work environment, describe physical restrictions associated with a particular job description, describe vision requirements associated with a particular job description, describe on-the-job risks associated with a particular job description, describe the physical characteristics required of a candidate for the job, and configure or specify a variety of parameters associated with a description or definition of the job for which a physical ability test event is being developed (e.g. see the user interface screenshots shown in FIGS. 6 through 11 and described below). This job description data can be gathered and processed by the job analysis module 210 and stored in database 150.

Additionally, the job analysis module 210 can use network 120 to access the plurality of external data sources 130 to obtain additional job-related data. For example, the job analysis module 210 can conduct network data searches for job descriptions and/or physical ability test data for physical ability tests designed for similar jobs in different geographical locations, similar jobs in different industries, similar jobs in different markets, and the like. The job analysis module 210 can also conduct network data searches for standards bodies, associations, or other organizations that publish applicable job-related data or physical ability test standards. This data can be retrieved and stored in the database 152. The job analysis module 210 can also use network 120 to access the plurality of external data sources 130 to obtain additional job-related legal data, information on PAT regulations, publications from legislative, administrative, judicial, or non-governmental sources. This data can be retrieved and stored in the database 153.

In a particular embodiment, these external data sources 130 can include individual or aggregated data sources as described above. Aggregated data sources can represent any of a variety of conventional sites or services that pull or receive jobs-related data items from various sources and collect the jobs-related data items in a local data collector. The local collector of a jobs-related data source 130 may then provide these jobs-related data items to host site 110 via the wide area data network 120.

Job analysis module 210 can be configured to interface with any of the external data sources 130 via wide area data network 120. Because of the variety of external data sources 130 providing data to job analysis module 210, the job analysis module 210 may need to manage the interface to the various external data sources 130. This data source interface or data management process performed by job analysis module 210 can include retaining meta information on each data item, document, or object received from a jobs-related data source 130. This retained meta information can include an identifier or address of the corresponding data item, document, or object received from a jobs-related data source 130, an identifier or address of the corresponding jobs-related data source 130, the timing or versioning associated with the data item, document, or object, including the time when the latest update of an item was received, and the like. This meta information related to the data items, documents, or objects received from external data sources 130 can be stored in database 150.

The data management process performed by job analysis module 210 can also include performing any transcoding, decompression, decryption, formatting, and/or the like that may be necessary to process and/or filter a particular data item received from a jobs-related data source 130 via host site 110. The data management process performed by job analysis module 210 can also include the fetching, staging, and processing of particular jobs-related data items retrieved from the external data sources 130.

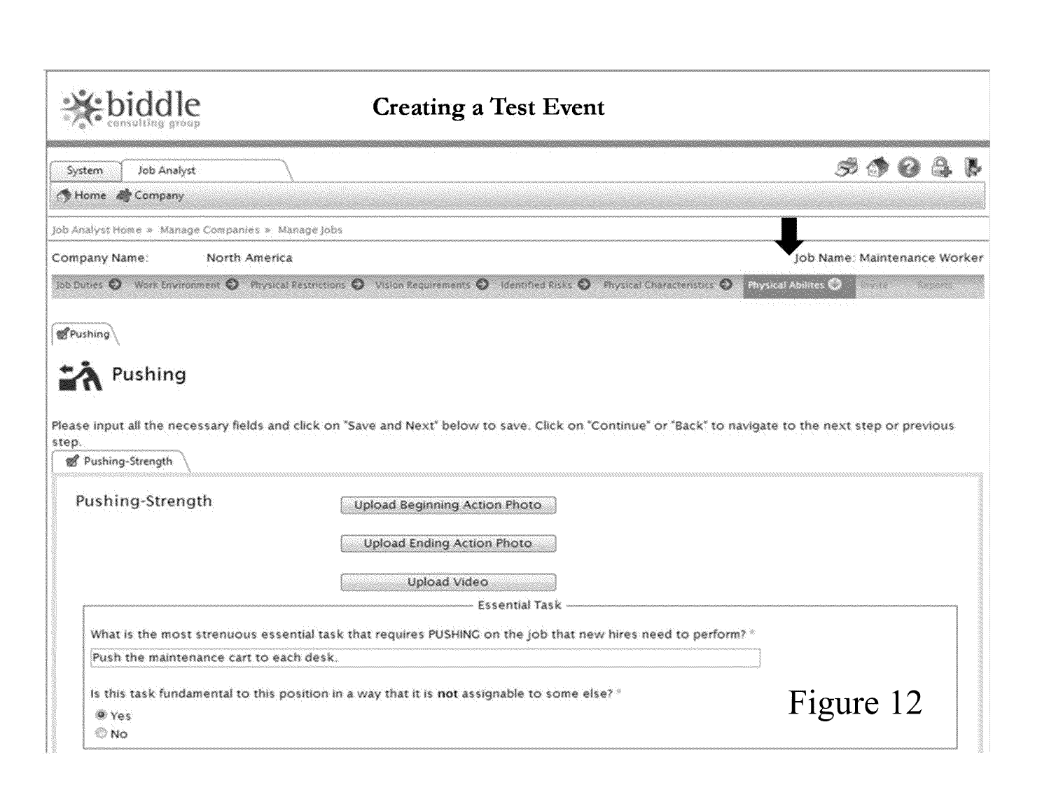

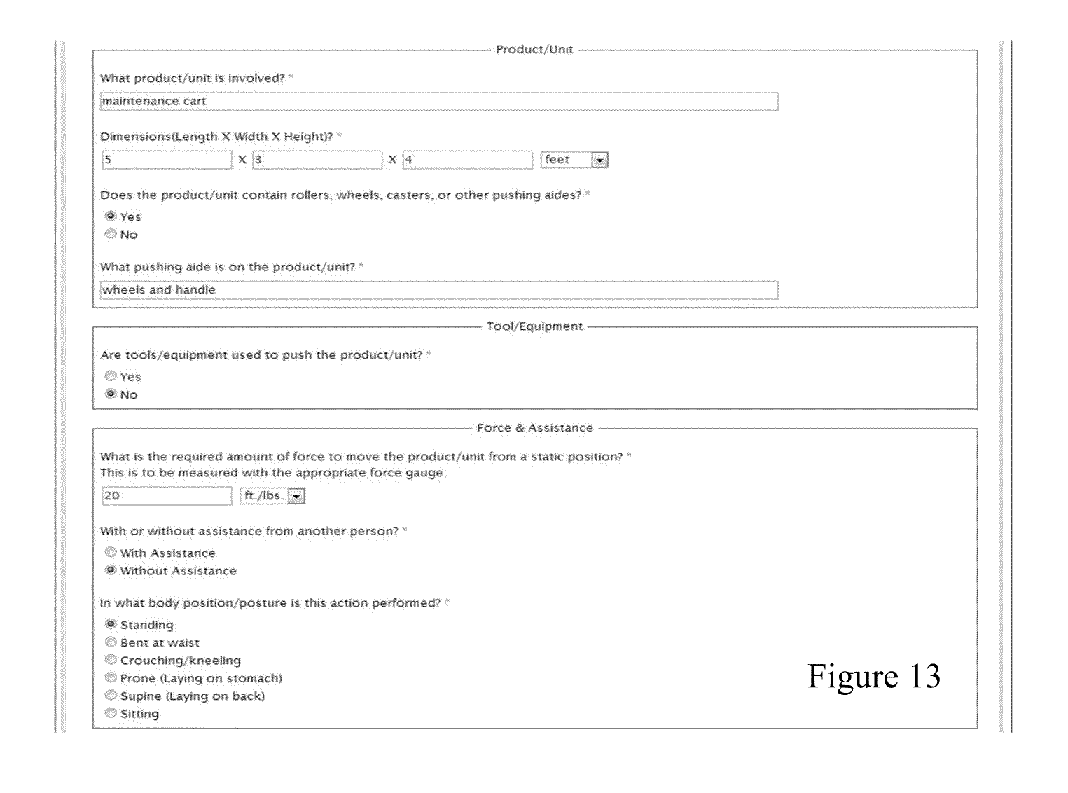

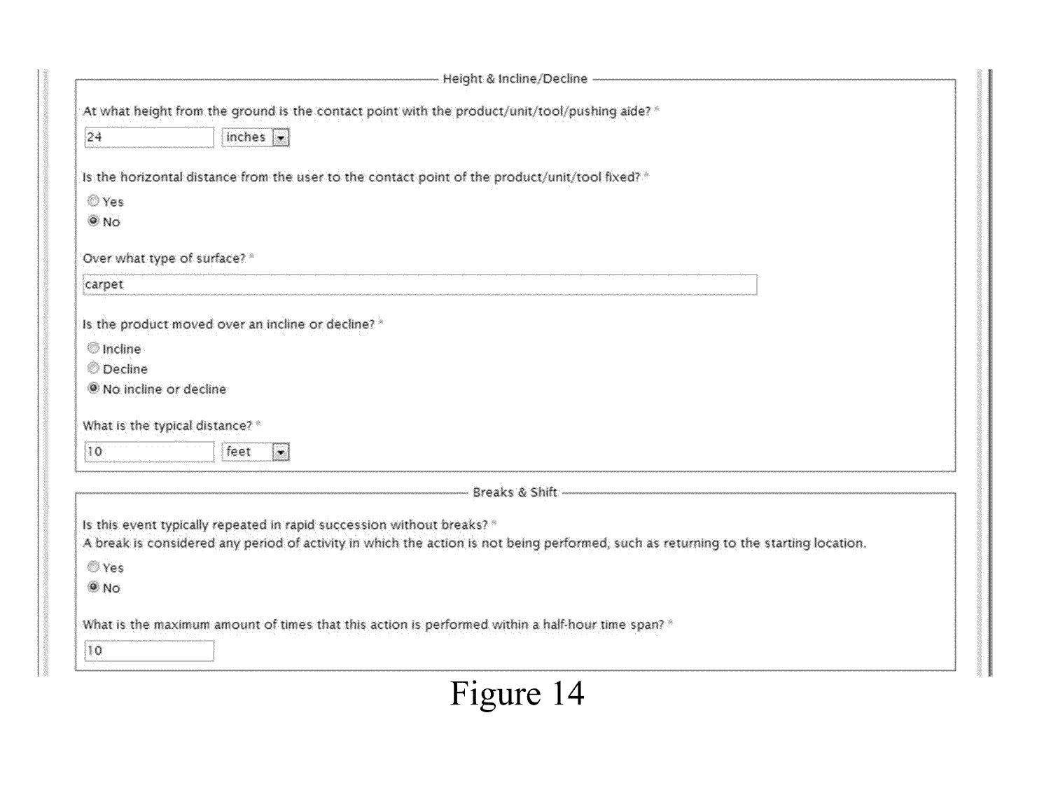

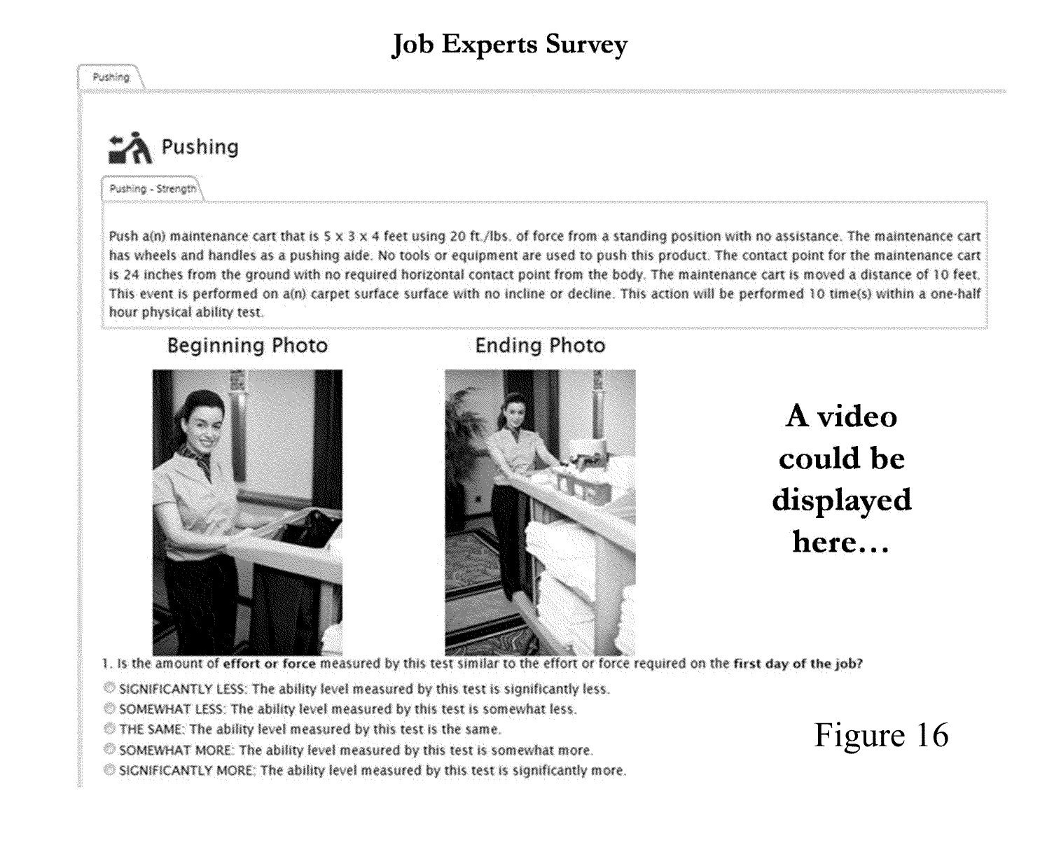

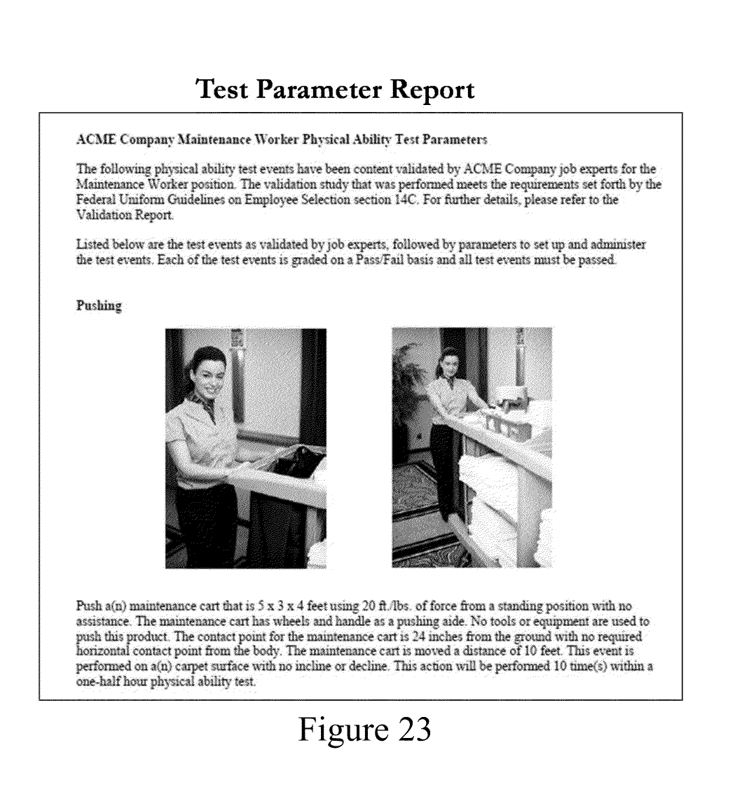

Referring again to FIG. 2, the PAT development system 200 of an example embodiment is shown to include a physical ability test (PAT) creation module 220. Once the job analysis module 210 has retrieved and processed the job-related data as described above, the PAT creation module 220 enables a user to create a particular proposed PAT event for a particular job description. In an example embodiment, the PAT creation module 220 can lead a user through a series of prompts or questions to gather the information necessary to create the proposed PAT event. An example of these prompts is illustrated in the sample screenshots illustrated in FIGS. 12 through 14. For example, the user can be queried regarding the physical abilities needed for the particular job description, such as pushing, pulling/dragging, gripping/pinching, lifting without carrying, lifting with carrying, climbing, ascending/descending steps, and walking, among other physical skills being tested. The input provided by the user can be captured and retained by the PAT creation module 220. Additionally, the user can be prompted for entry and upload of any photos, images, videos, or the like that may be related to the PAT test event being created. Images or videos are particularly helpful to enable a job analyst or job expert to visualize the actual environment in which the job activities will occur. The PAT creation module 220 can use the input provided by the user and the provided images or videos to create an illustrated narrative that describes the proposed physical ability test event. FIGS. 16 and 23 show an example of the illustrated narrative created by the PAT creation module 220 of an example embodiment. Thus, as configured and directed by the user via the computer-generated user interface, the PAT creation module 220 of an example embodiment described herein can automatically create a proposed physical ability test event based on prompted user input and the information produced by the job analysis module 210 as described above. Although the proposed physical ability test event created by the PAT creation module 220 can be considered a complete and executable test event, the example embodiments described herein perform additional validation processing operations to ensure the proposed physical ability test event is safe, effective, and legally compliant. These validation processing operations are managed by the physical ability test (PAT) validation module 230 described below.

The various example embodiments described herein can provide a variety of unique features including at least: 1) dynamic lift reserve calculation, and 2) dynamic weight variation. The PAT creation module 220 of an example embodiment can implement these features. An example embodiment as described herein can automatically apply a dynamic lift reserve calculation to accommodate and adjust for non-optimal posture or objects. An example embodiment can compare a current lifting task to the National Institute for Occupational Safety and Health (NIOSH) Lifting Index. The NIOSH Lifting Index relates to a standardized method to assess risk of low-back disorders in jobs with repeated lifting or other physical activity. The method of an example embodiment comprises at least two primary parameters, the recommended weight limit (RWL) and the lifting index (LI). The RWL is defined for a specific set of task conditions as the weight of the load that nearly all healthy workers could perform over a substantial period of time (e.g. up to 8 hours) without an increased risk of developing lifting-related low back pain. The lifting index (LI) is the ratio of the actual load weight and the recommended weight limit. The LI is related to jobs with similar lifting tasks. An example embodiment can automatically compare a current lifting or other physical activity task for a particular job to the NIOSH lifting index for the job category.

An example embodiment can also automatically apply dynamic weight variation to add additional weight to or subtract weight from a test event based upon a conditional standard error of measurement (CSEM). The dynamic weight variation can be applied as contiguous activity without breaks and/or in a manner that can be performance differentiating. The application of dynamic weight variation enables a particular physical ability test event to more clearly differentiate the subjects being tested.

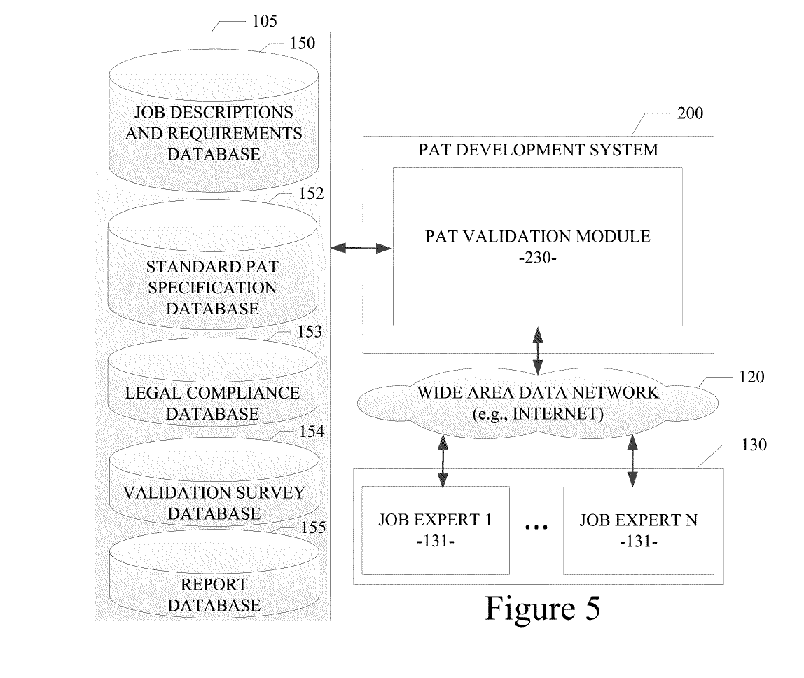

The PAT development system 200 of an example embodiment includes a physical ability test (PAT) validation module 230. Referring now to FIG. 5, a detail of the physical ability test (PAT) validation module 230 of an example embodiment is illustrated. As shown, physical ability test (PAT) validation module 230 is in data communication via network 120 with a plurality of external data sources 130, one or more portions of database 105, and the other processing modules of the PAT development system 200 (not shown in FIG. 5). The plurality of external data sources 130 can include a plurality of job experts 131. In general, the PAT validation module 230 is responsible for performing validation processing operations to ensure the proposed physical ability test event is safe, effective, and legally compliant. The validation processing operations in an example embodiment include generating a validation survey based on the proposed physical ability test event, sending validation survey invites to appropriate job experts, and receiving and processing feedback from the surveyed job experts. The job expert feedback is an important component supporting the legal compliance of the proposed physical ability test event with applicable regulations and guidelines. The validation survey is used to enable the job experts to vet and validate the proposed physical ability test event. The job experts surveyed by the PAT validation module 230 represent a set of qualified job experts with an expertise in the job category associated with the particular job offering. The job experts can be in data communication with the PAT validation module 230 via network 120 and client application software, an email or chat client, or other conventional network communication means.

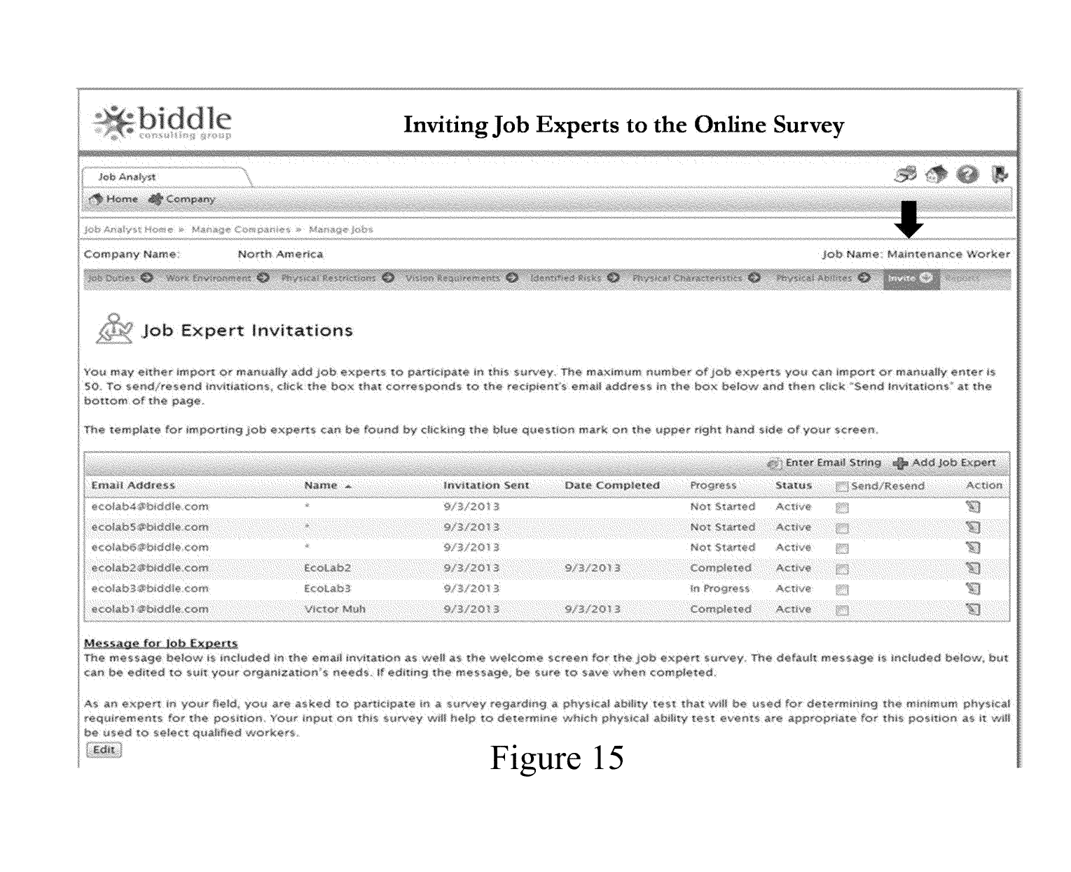

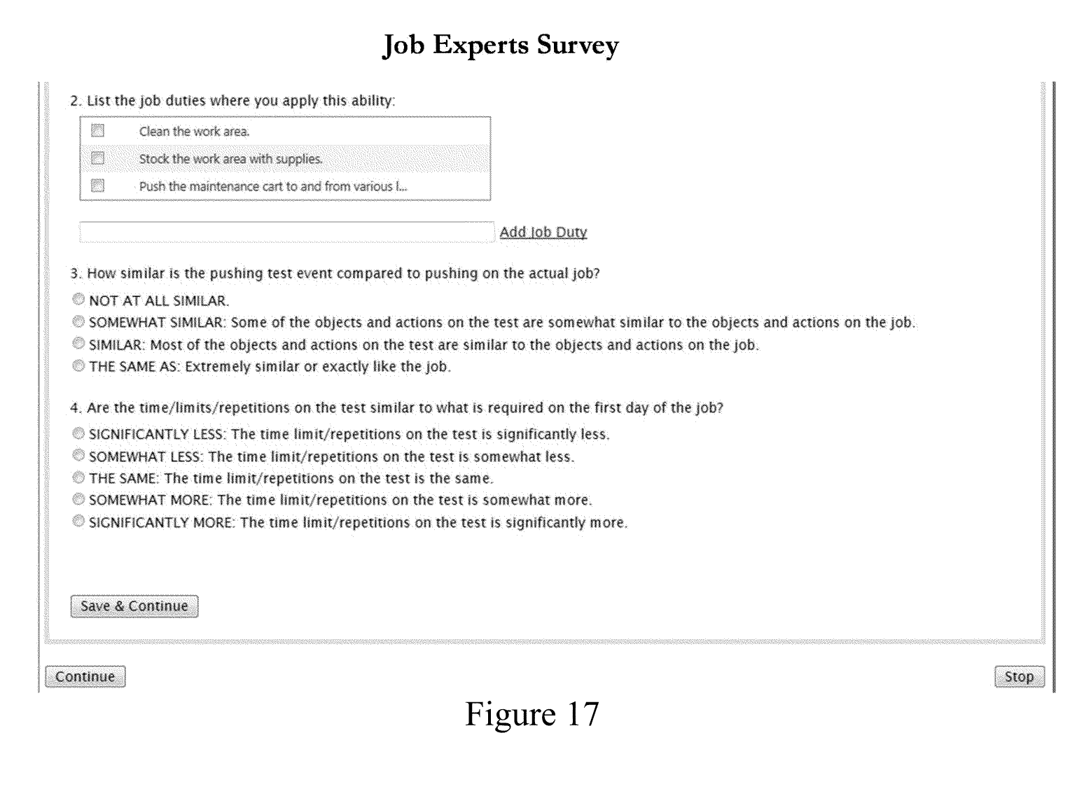

Once the PAT creation module 220 of an example embodiment automatically creates the proposed physical ability test event as described above, the PAT validation module 230 can produce a validation survey, which is automatically sent with an invitation to appropriate job experts. An example validation survey and invitation in an example embodiment is shown in FIGS. 15 through 17. The validation survey can include a sample of the illustrated narrative created by the PAT creation module 220 and a set of prompts or questions for the job experts to provide feedback relative to the proposed physical ability test event. The validation survey and invitation can be electronically sent to the set of job experts via network 120 using conventional email, text messaging, chat, or other data communication means. Similarly, the job expert feedback in response to the validation survey and invitation can be received by the PAT validation module 230 from the set of job experts via network 120.

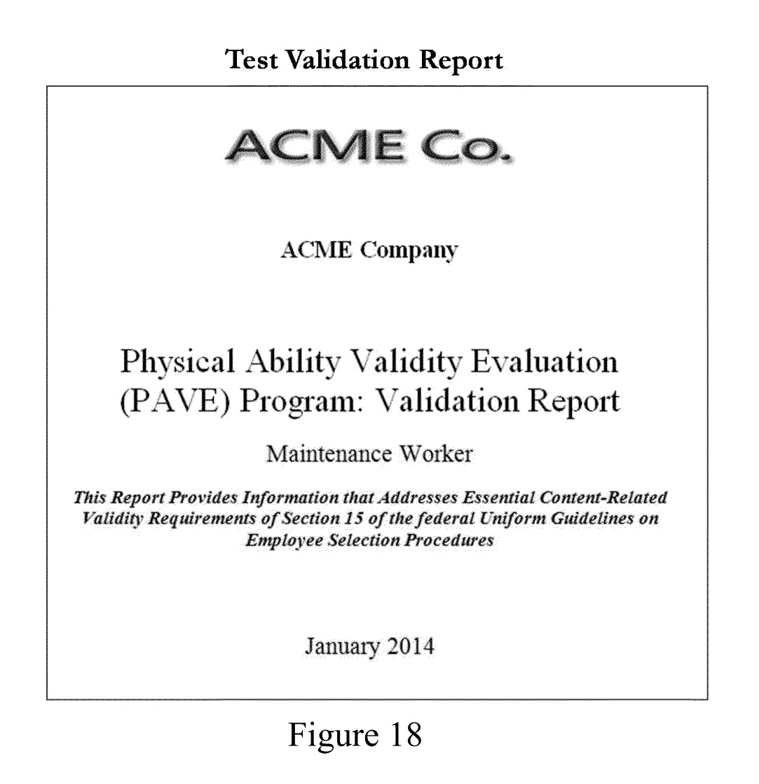

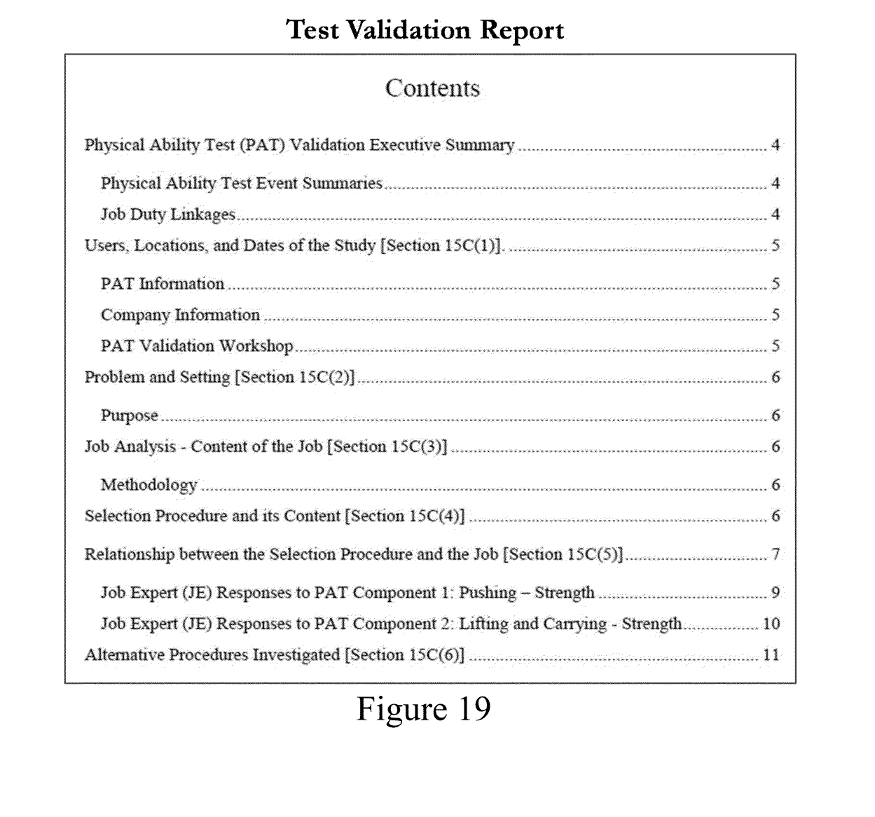

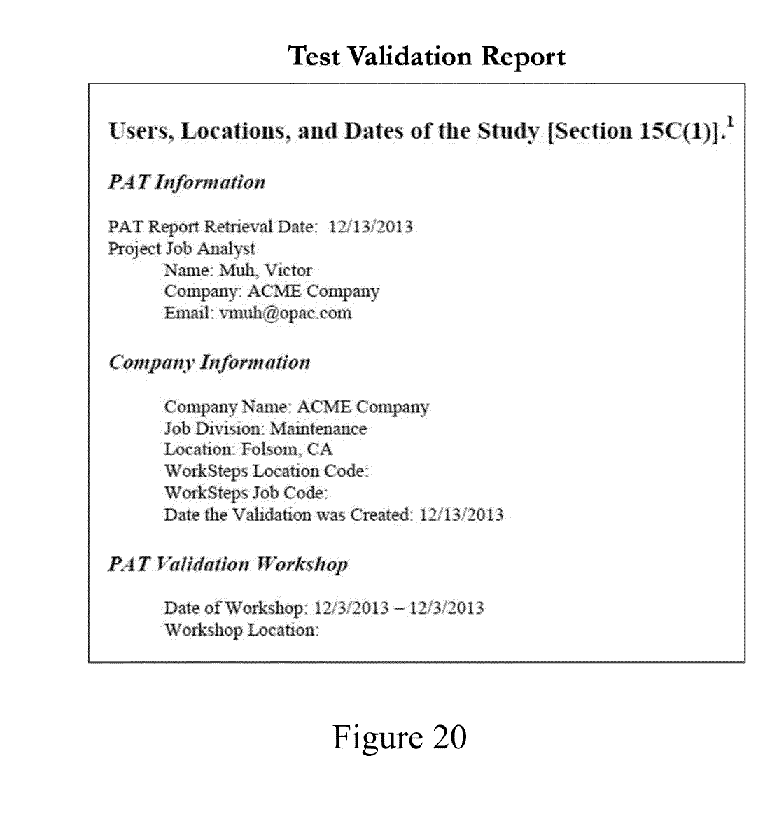

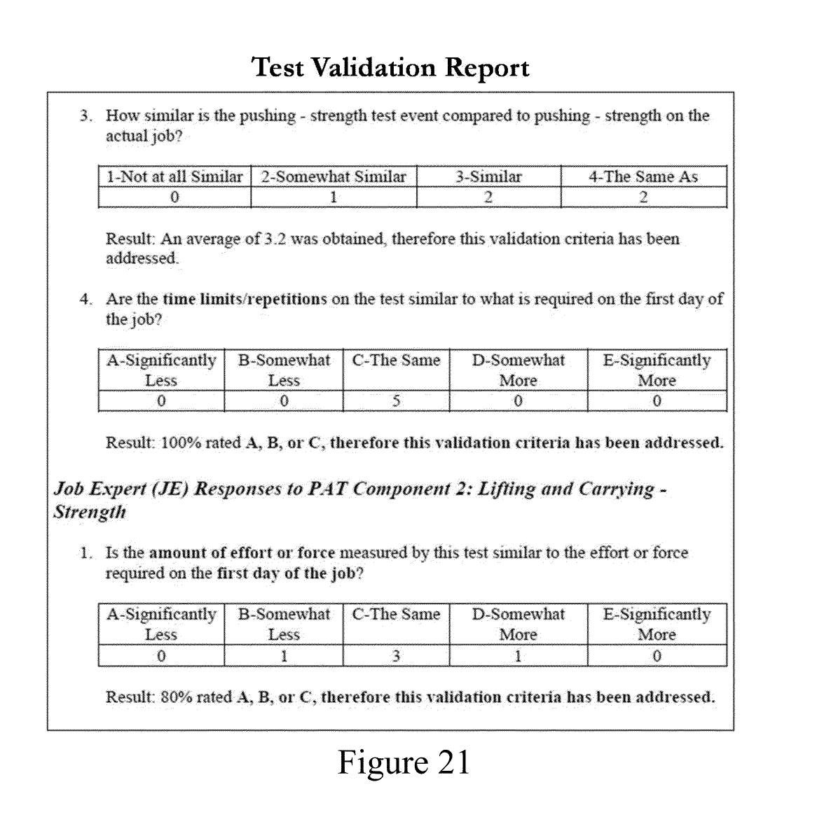

Referring again to FIG. 2, the PAT development system 200 of an example embodiment is shown to include a validation report generator 240 and a parameter report generator 250. After the validation survey is completed and the job expert feedback is retrieved, an example embodiment can dynamically and automatically generate a validation report including the feedback from the surveyed set of job experts. The validation report generator 240 is responsible for generating the validation report. An example validation report in an example embodiment is shown in FIGS. 18 through 21. As shown in the example validation report, the validation report includes test and survey identifying information and a summary of the results submitted by the job experts in response to the validation survey. The validation report is also automatically configured to include all of the information, supporting documentation, metrics, and analytics necessary to comply with all applicable rules, regulations, and guidelines related to the employment assessment test event as determined by the job analysis module 210 described above. As a result, the PAT event is legally compliant and all supporting documentation is automatically generated.

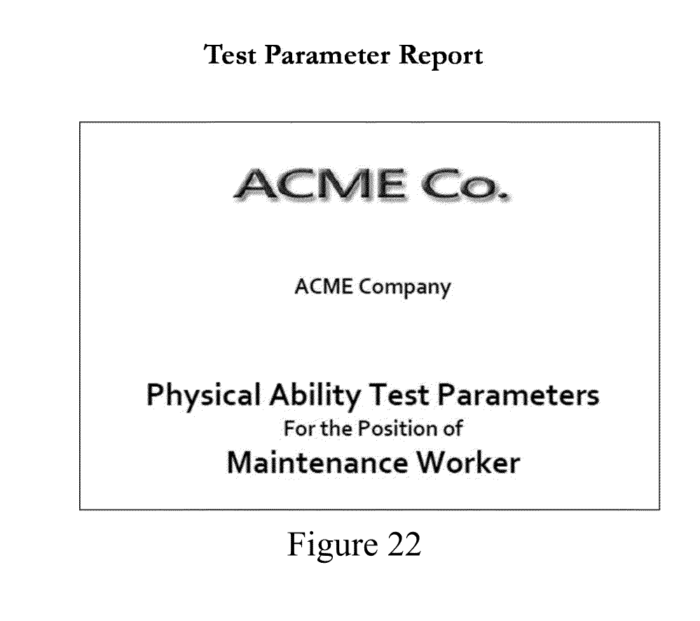

The example embodiment also includes a parameter report generator 250. The parameter report generator 250 is responsible for generating a parameter report, which defines the specifics of the PAT event. A job analyst can use the parameter report to execute a particular PAT event for a job applicant. An example parameter report in an example embodiment is shown in FIGS. 22 through 23. As shown in the example parameter report, the parameter report includes test and survey identifying information, specific test parameters, and the illustrated narrative created by the PAT creation module 220. It will be apparent to those of ordinary skill in the art in view of the disclosure herein that the parameter report in a particular embodiment can include additional information to facilitate execution of the PAT event by a job analyst, an employer, or the like.

Referring again to FIG. 2, the PAT development system 200 of an example embodiment is shown to include a hosted application management module 260. As one option, the PAT development system 200, or a portion thereof, can be downloaded to a user device 141 of user platform 140 and executed locally on a user device 141. The downloading of the PAT development system 200 application can be accomplished using conventional software downloading functionality. As a second option, the PAT development system 200 can be hosted by the host site 110 and executed remotely, from the user's perspective, on host system 110. In one embodiment, the PAT development system 200 can be implemented as a service in a service oriented architecture (SOA). In any case, the functionality performed by the PAT development system 200 is as described herein, whether the application is executed locally or remotely, relative to the user.

Referring again to FIG. 2, the PAT development system 200 of an example embodiment is shown to include a user interface management module 270. The user interface management module 270 handles the presentation of various user interface displays, display screens, windows, frames, or the like. In an alternative embodiment, the user interface can be implemented as a series of web pages. In another alternative embodiment, the user interface can be implemented as a series display screens on a mobile device as implemented by a mobile application or app. In yet another alternative embodiment, the user interface can be implemented as a series of voice command/responses implemented using conventional voice recognition and voice synthesis technology on a mobile device as implemented by a mobile application or app. These various user interface displays are provided to present information and images to a user of the PAT development system 200 of an example embodiment. The user interface management module 270 also receives user inputs and configures the various user interface displays in conformity with the user inputs, command selections, and the like.

Although the various user interface displays provided by the user interface management module 270 are nearly infinitely varied, several sample user interface sequences are provided herein and in the corresponding figures to describe various features of the disclosed embodiments. These sample user interface displays and sequences are illustrated in the accompanying figures.

Referring again to FIG. 2, as described above, the PAT development system 200, or a portion thereof, can be downloaded to a user device 141 of user platform 140 or executed on host system 110. The PAT development system 200 may communicate and transfer data and information in the network ecosystem via a wide area data network (e.g., the Internet) 120. In this manner, the PAT development system 200 may communicate and transfer data and information with a user of a user device 141 of user platform 140. In general, the user can interact with the PAT development system 200 via a graphical user interface (GUI) implemented in an example embodiment as a set of webpages or application display screens described in more detail below.

Referring now to FIGS. 6 through 17, several sample user interface screen snapshots and sequences are illustrated. These sample user interface sequences illustrate various features, structures, and operations of various example embodiments. Referring to FIG. 6, for each of these sample user interface sequences, a user interface screen 100, window, or display image is presented on a user/client device 141. The user interface 100 typically includes a set of information and command options for selection by a user. Each of these user interface screens 100 shown in FIGS. 6 through 17 are described next as part of the generation and execution of a sample proposed physical ability test event by the PAT development system 200 of an example embodiment.

Referring now to FIG. 6, the sample user interface screen 100 illustrates the result after a user has launched the PAT development system 200 of an example embodiment. As shown, the user is initially presented with a set of tabs 102 corresponding to various sets of command options. In the particular sample embodiment, the command tab denoted as ‘Job Duties’ has been selected by the user. The Job Duties command option allows the user to configure and specify the details of a particular job description corresponding to a job type or job category for which the user is creating a physical ability test event.

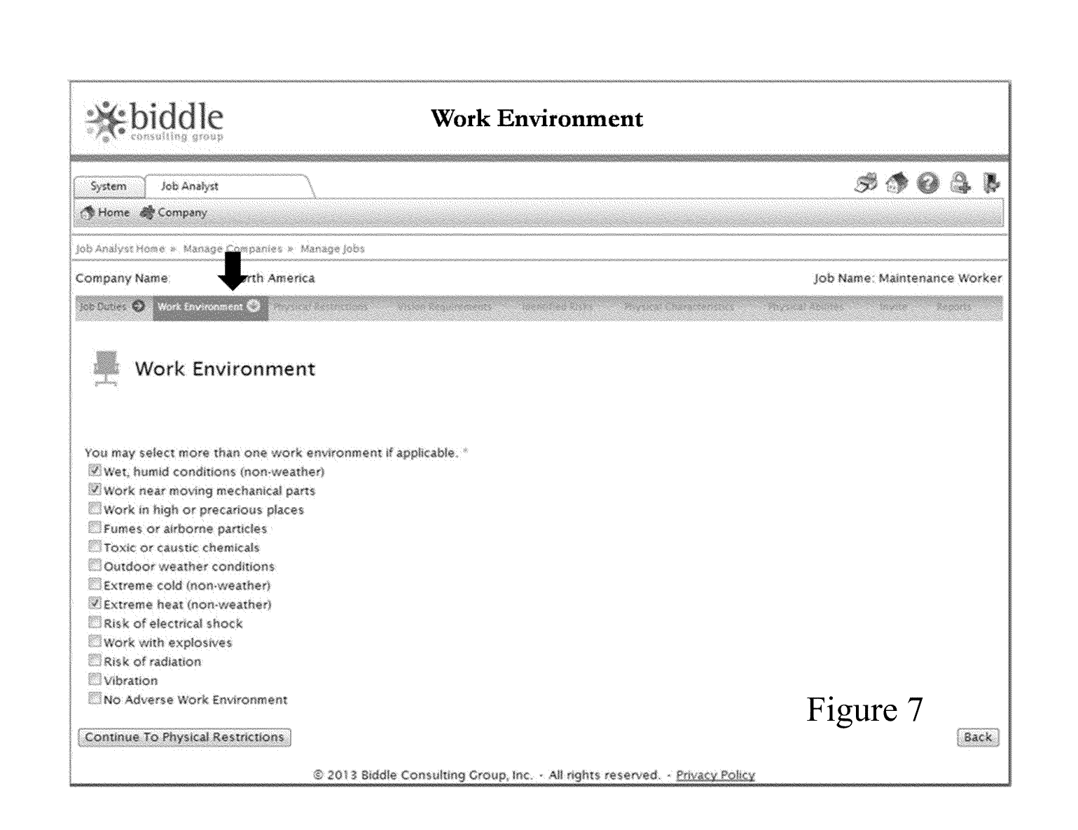

Referring now to FIG. 7, the sample user interface screen illustrates the result after a user has selected the “Work Environment” command tab in the PAT development system 200 of an example embodiment. The Work Environment command option allows the user to configure and specify the details of a particular work or job environment corresponding to a job type or job category for which the user is creating a physical ability test event.

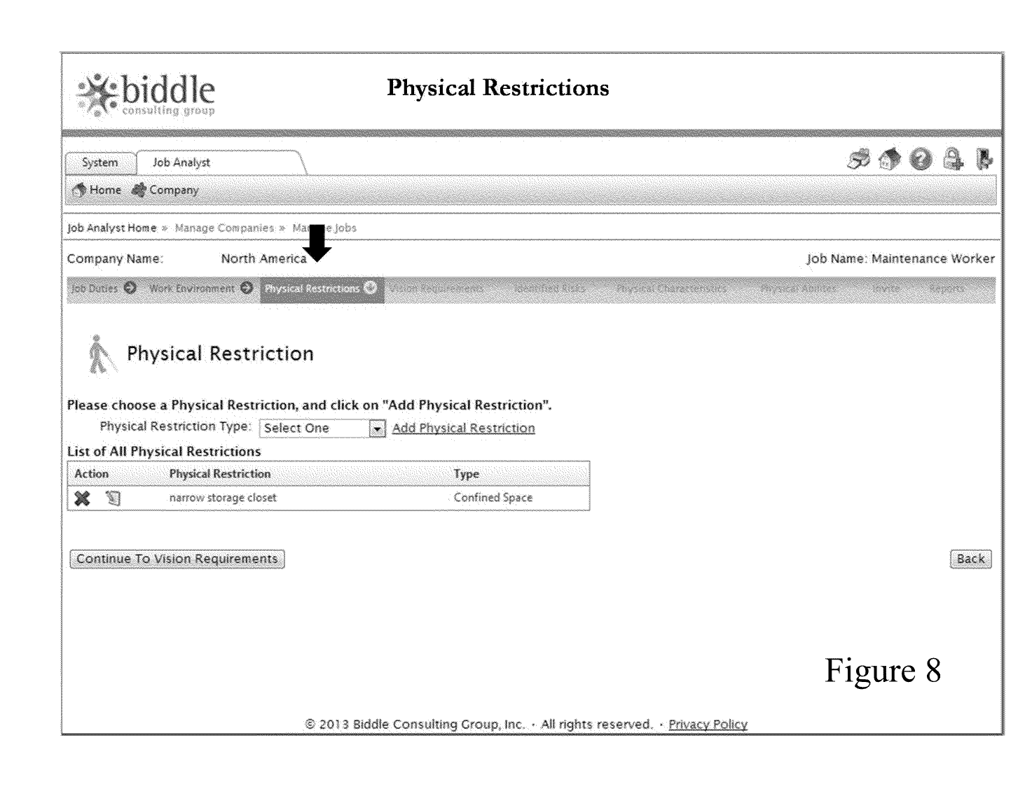

Referring now to FIG. 8, the sample user interface screen illustrates the result after a user has selected the “Physical Restrictions” command tab in the PAT development system 200 of an example embodiment. The Physical Restrictions command option allows the user to configure and specify the details of particular physical restrictions corresponding to a job type or job category for which the user is creating a physical ability test event.

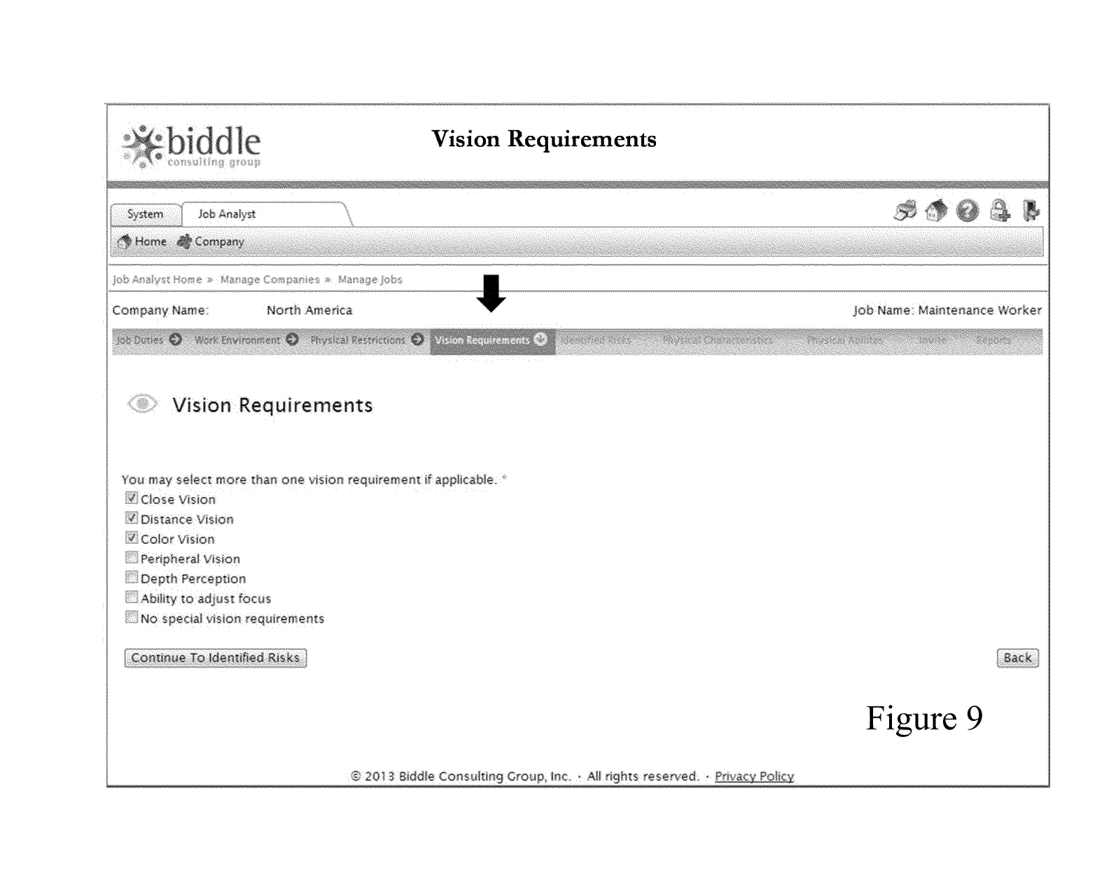

Referring now to FIG. 9, the sample user interface screen illustrates the result after a user has selected the “Vision Requirements” command tab in the PAT development system 200 of an example embodiment. The Vision Requirements command option allows the user to configure and specify the details of particular vision requirements corresponding to a job type or job category for which the user is creating a physical ability test event.

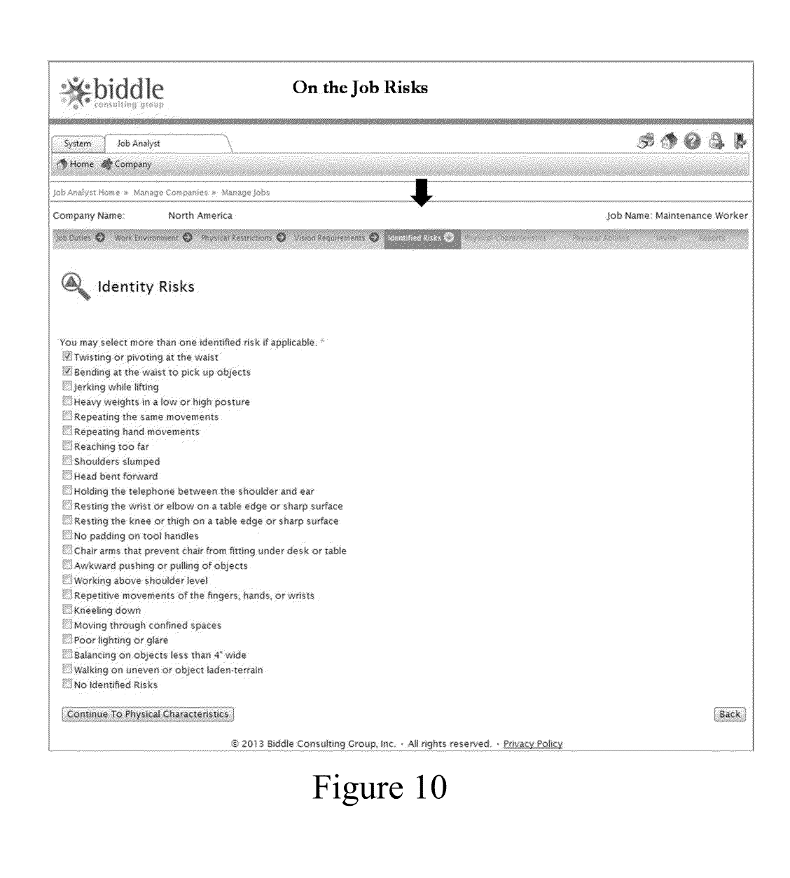

Referring now to FIG. 10, the sample user interface screen illustrates the result after a user has selected the “On the Job Risks” command tab in the PAT development system 200 of an example embodiment. The On the Job Risks command option allows the user to configure and specify the details of the risks associated with a particular job or work environment corresponding to a job type or job category for which the user is creating a physical ability test event.

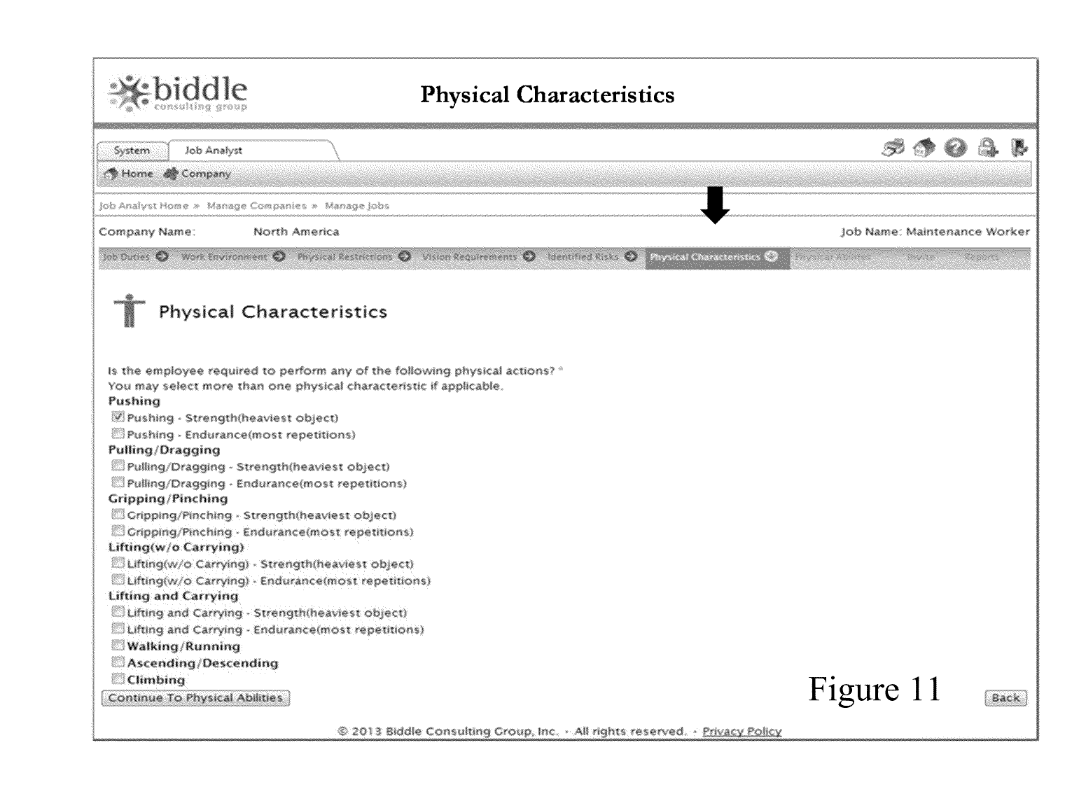

Referring now to FIG. 11, the sample user interface screen illustrates the result after a user has selected the “Physical Characteristics” command tab in the PAT development system 200 of an example embodiment. The Physical Characteristics command option allows the user to configure and specify the details of the particular physical characteristics corresponding to a job type or job category for which the user is creating a physical ability test event.

Referring now to FIGS. 12 through 14, the sample user interface screens illustrate the result after a user has selected the “Creating a Test Event” command tab in the PAT development system 200 of an example embodiment. The PAT event creation process was described above in connection with PAT creation module 220.

Referring now to FIGS. 15 through 17, the sample user interface screens illustrate the result after a user has selected the “Inviting Job Experts to the Online Survey” command tab in the PAT development system 200 of an example embodiment. The job expert invitation and PAT event validation process was described above in connection with PAT validation module 230.

Referring now to FIGS. 18 through 21, a sample Test Validation Report automatically created by the PAT development system 200 of an example embodiment is illustrated. The Test Validation Report generation process in an example embodiment was described above in connection with the validation report generator 240.

Referring now to FIGS. 22 through 23, a sample Test Parameter Report automatically created by the PAT development system 200 of an example embodiment is illustrated. The Test Parameter Report generation process in an example embodiment was described above in connection with the parameter report generator 250.

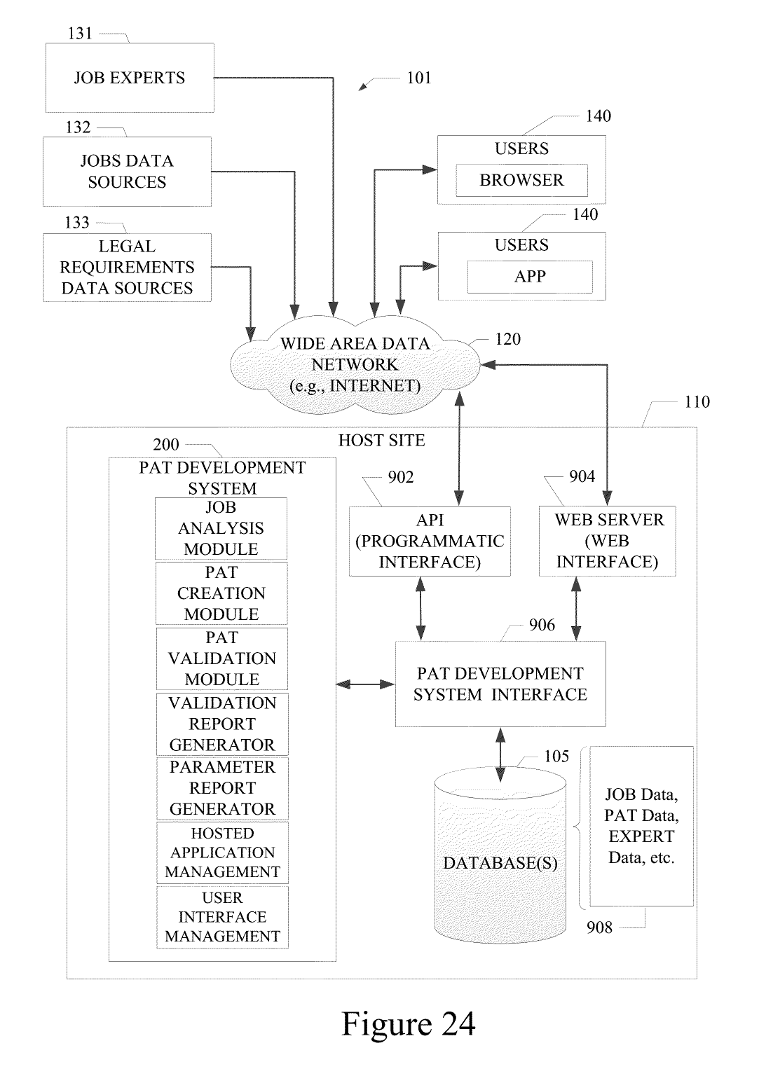

Referring now to FIG. 24, another example embodiment 101 of a networked system in which various embodiments may operate is illustrated. In the embodiment illustrated, the host site 110 is shown to include the PAT development system 200. The PAT development system 200 is shown to include the functional components 210-270, as described above. In a particular embodiment, the host site 110 may also include a web server 904, having a web interface with which users may interact with the host site 110 via a user interface or web interface. The host site 110 may also include an application programming interface (API) 902 with which the host site 110 may interact with other network entities on a programmatic or automated data transfer level. The API 902 and web interface 904 may be configured to interact with the PAT development system 200 either directly or via an interface 906. The PAT development system 200 may be configured to access a data storage device 105 either directly or via the interface 906.

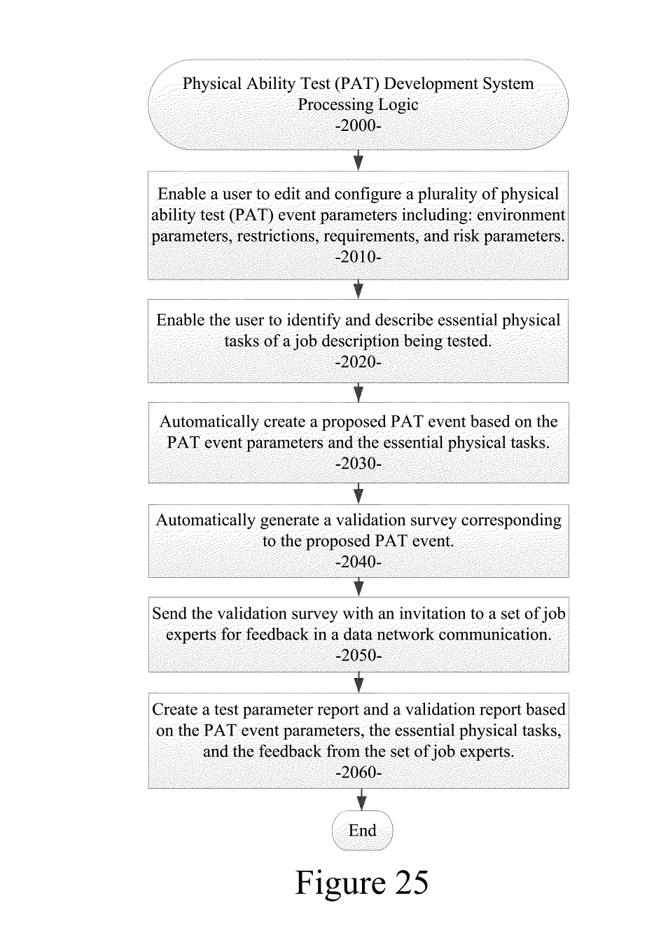

Referring now to FIG. 25, a processing flow diagram illustrates an example embodiment of a PAT development system 200 as described herein. The method 2000 of an example embodiment includes: enabling, by use of a computing device, a user to edit and configure a plurality of physical ability test (PAT) event parameters including: environment parameters, restrictions, requirements, and risk parameters (processing block 2010); enabling, by use of the computing device, the user to identify and describe essential physical tasks of a job description being tested (processing block 2020); automatically creating a proposed PAT event based on the PAT event parameters and the essential physical tasks (processing block 2030); automatically generating a validation survey corresponding to the proposed PAT event (processing block 2040); sending the validation survey with an invitation to a set of job experts for feedback in a data network communication (processing block 2050); and creating a test parameter report and a validation report based on the PAT event parameters, the essential physical tasks, and the feedback from the set of job experts (processing block 2060).

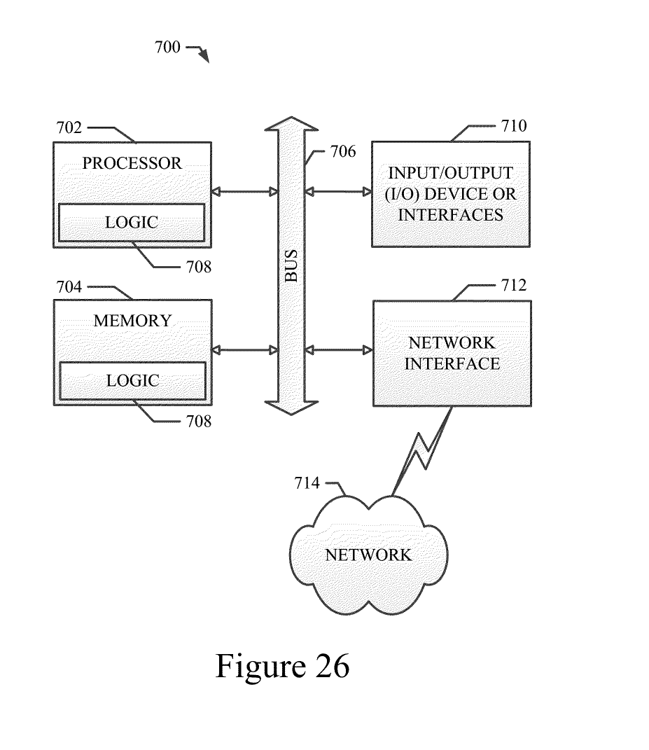

FIG. 26 shows a diagrammatic representation of a machine in the example form of a mobile computing and/or communication system 700 within which a set of instructions when executed and/or processing logic when activated may cause the machine to perform any one or more of the methodologies described and/or claimed herein. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a personal computer (PC), a laptop computer, a tablet computing system, a Personal Digital Assistant (PDA), a cellular telephone, a smartphone, a web appliance, a set-top box (STB), a network router, switch or bridge, or any machine capable of executing a set of instructions (sequential or otherwise) or activating processing logic that specify actions to be taken by that machine. Further, while only a single machine is illustrated, the term “machine” can also be taken to include any collection of machines that individually or jointly execute a set (or multiple sets) of instructions or processing logic to perform any one or more of the methodologies described and/or claimed herein.

The example mobile computing and/or communication system 700 includes a data processor 702 (e.g., a System-on-a-Chip (SoC), general processing core, graphics core, and optionally other processing logic) and a memory 704, which can communicate with each other via a bus or other data transfer system 706. The mobile computing and/or communication system 700 may further include various input/output (I/O) devices and/or interfaces 710, such as a touchscreen display, an audio jack, and optionally a network interface 712. In an example embodiment, the network interface 712 can include one or more radio transceivers configured for compatibility with any one or more standard wireless and/or cellular protocols or access technologies (e.g., 2nd (2G), 2.5, 3rd (3G), 4th (4G) generation, and future generation radio access for cellular systems, Global System for Mobile communication (GSM), General Packet Radio Services (GPRS), Enhanced Data GSM Environment (EDGE), Wideband Code Division Multiple Access (WCDMA), LTE, CDMA2000, WLAN, Wireless Router (WR) mesh, and the like). Network interface 712 may also be configured for use with various other wired and/or wireless communication protocols, including TCP/IP, UDP, SIP, SMS, RTP, WAP, CDMA, TDMA, UMTS, UWB, WiFi, WiMax, Bluetooth, IEEE 802.11x, and the like. In essence, network interface 712 may include or support virtually any wired and/or wireless communication mechanisms by which information may travel between the mobile computing and/or communication system 700 and another computing or communication system via network 714.

The memory 704 can represent a machine-readable medium on which is stored one or more sets of instructions, software, firmware, or other processing logic (e.g., logic 708) embodying any one or more of the methodologies or functions described and/or claimed herein. The logic 708, or a portion thereof, may also reside, completely or at least partially within the processor 702 during execution thereof by the mobile computing and/or communication system 700. As such, the memory 704 and the processor 702 may also constitute machine-readable media. The logic 708, or a portion thereof, may also be configured as processing logic or logic, at least a portion of which is partially implemented in hardware. The logic 708, or a portion thereof, may further be transmitted or received over a network 714 via the network interface 712. While the machine-readable medium of an example embodiment can be a single medium, the term “machine-readable medium” should be taken to include a single non-transitory medium or multiple non-transitory media (e.g., a centralized or distributed database, and/or associated caches and computing systems) that stores the one or more sets of instructions. The term “machine-readable medium” can also be taken to include any non-transitory medium that is capable of storing, encoding or carrying a set of instructions for execution by the machine and that cause the machine to perform any one or more of the methodologies of the various embodiments, or that is capable of storing, encoding or carrying data structures utilized by or associated with such a set of instructions. The term “machine-readable medium” can accordingly be taken to include, but not be limited to, solid-state memories, optical media, and magnetic media.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A computer-implemented method comprising:
enabling, by use of a computing device, a user to edit and configure a plurality of physical ability test (PAT) event parameters including: environment parameters, restrictions, requirements, and risk parameters;
enabling, by use of the computing device, the user to identify and describe essential physical tasks of a job description being tested;
automatically creating a proposed PAT event based on the PAT event parameters and the essential physical tasks;
automatically generating a validation survey corresponding to the proposed PAT event;
sending the validation survey with an invitation to a set of job experts for feedback in a data network communication; and
creating a test parameter report and a validation report based on the PAT event parameters, the essential physical tasks, and the feedback from the set of job experts.

2. The method as claimed in claim 1 wherein the environment parameters include information specifying a type of location in which the tasks of the job description being tested are typically performed.

3. The method as claimed in claim 1 wherein the restrictions include information specifying a capability or physical ability a qualified job applicant must possess.

4. The method as claimed in claim 1 wherein the risk parameters include information specifying a type of risk associated with the tasks of the job description being tested.

5. The method as claimed in claim 1 wherein the essential physical tasks include information specifying types of physical activities required for the job description being tested.

6. The method as claimed in claim 1 wherein automatically creating a proposed PAT event includes automatically applying a dynamic lift reserve calculation.

7. The method as claimed in claim 1 wherein automatically creating a proposed PAT event includes automatically applying a dynamic weight variation.

8. The method as claimed in claim 1 wherein sending the validation survey with an invitation to a set of job experts for feedback in a data network communication includes initiating a data network communication of a type from the group consisting of: email, text messaging, and chat.

9. A system comprising:
a processor;
a database, in data communication with the processor, for storage of job-related data; and
a physical ability test (PAT) development module, executable by the processor, to:
enable a user to edit and configure a plurality of physical ability test (PAT) event parameters including: environment parameters, restrictions, requirements, and risk parameters;
enable the user to identify and describe essential physical tasks of a job description being tested;
automatically create a proposed PAT event based on the PAT event parameters and the essential physical tasks;
automatically generate a validation survey corresponding to the proposed PAT event;
send the validation survey with an invitation to a set of job experts for feedback in a data network communication; and
create a test parameter report and a validation report based on the PAT event parameters, the essential physical tasks, and the feedback from the set of job experts.

10. The system as claimed in claim 9 wherein the environment parameters include information specifying a type of location in which the tasks of the job description being tested are typically performed.

11. The system as claimed in claim 9 wherein the restrictions include information specifying a capability or physical ability a qualified job applicant must possess.

12. The system as claimed in claim 9 wherein the risk parameters include information specifying a type of risk associated with the tasks of the job description being tested.

13. The system as claimed in claim 9 wherein the essential physical tasks include information specifying types of physical activities required for the job description being tested.

14. The system as claimed in claim 9 being further configured to automatically create a proposed PAT event includes automatically applying a dynamic lift reserve calculation.

15. The system as claimed in claim 9 being further configured to automatically create a proposed PAT event includes automatically applying a dynamic weight variation.

16. The system as claimed in claim 9 being further configured to automatically initiate a data network communication of a type from the group consisting of: email, text messaging, and chat.

17. A non-transitory machine-useable storage medium embodying instructions which, when executed by a machine, cause the machine to:
enable a user to edit and configure a plurality of physical ability test (PAT) event parameters including: environment parameters, restrictions, requirements, and risk parameters;
enable the user to identify and describe essential physical tasks of a job description being tested;
automatically create a proposed PAT event based on the PAT event parameters and the essential physical tasks;
automatically generate a validation survey corresponding to the proposed PAT event;
send the validation survey with an invitation to a set of job experts for feedback in a data network communication; and
create a test parameter report and a validation report based on the PAT event parameters, the essential physical tasks, and the feedback from the set of job experts.

18. The non-transitory machine-useable storage medium as claimed in claim 17 being further configured to automatically create a proposed PAT event includes automatically applying a dynamic lift reserve calculation.

19. The non-transitory machine-useable storage medium as claimed in claim 17 being further configured to automatically create a proposed PAT event includes automatically applying a dynamic weight variation.

20. The non-transitory machine-useable storage medium as claimed in claim 17 being further configured to automatically initiate a data network communication of a type from the group consisting of: email, text messaging, and chat.
